(19) United States
(12) Patent Application Publication
(10) Pub. No.: US 2016/0313311 A1
(43) Pub. Date: Oct. 27, 2016

(54) LABEL-FREE DETECTION OF MACROMOLECULES BINDING TO COMPOUND LIBRARIES

(71) Applicant: The Regents of the University of California, Oakland, CA (US)

(72) Inventors: Michael Schlein, Sacramento, CA (US); Kit S. Lam, Sacramento, CA (US)

(21) Appl. No.: 15/136,783

(22) Filed: Apr. 22, 2016

Related U.S. Application Data
(60) Provisional application No. 62/152,608, filed on Apr. 24, 2015.

(51) Int. Cl. G01N 33/537; G01N 27/447; G01N 33/53

(52) U.S. Cl. CPC G01N 33/537; G01N 33/5308; G01N 27/44782

(57) ABSTRACT

A one-bead-one-compound (OBOC) assay technique and associated components and kits are disclosed herein. With the described assay, target macromolecules that are bound to compound beads, and the compounds can be identified and separated without labeling the compounds.

### CROSS-REFERENCE TO RELATED APPLICATION

This application claims priority under 35 U.S.C. §119(e) to U.S. Provisional Application No. 62/152,608, filed Apr. 24, 2015, the content of which is hereby incorporated by reference in its entirety.

### FIELD OF THE INVENTION

The present invention relates generally to combinatorial chemistry and specifically to identification of lead compounds using an improved one-bead-one-compound technique.

### BACKGROUND

One-Bead-One-Compound (OBOC) techniques have existed for approximately two decades and have been well characterized to discover useful drugs to treat disease by screening for new compounds. OBOC libraries have been used to identify macromolecule-binding compounds for a nearly limitless number of different types of macromolecules, including macromolecule-binding compounds useful against various cancers (Lam, K. S. et al. (1997) Chem. Rev. 97:411-448). An OBOC library may include millions of polymeric beads, with each bead display multiple copies of the same but unique compound. Each bead-bound compound is referred to as a compound bead. In various OBOC screening methods, a target macromolecule is introduced to an OBOC library and detection techniques are used to determine which compound beads positively bind with the target macromolecule. Compounds displayed on these positive beads are often deemed to be pharmacologically or chemically promising compounds and are referred to as lead compounds.

To detect binding of a compound bead to a target macromolecule such as protein, DNA, and RNA, conventional OBOC screening methods have attached a visual label that emits color and/or fluorescence to the target macromolecule to make positive macromolecule-bound compound beads stand out (Lam, K. S. et al. (1991) Nature 354:82). For example, a common way of detecting binding of a peptide or small molecule bead to a target macromolecule is by biotinylating that target macromolecule and then probing for the biotin using an anti-biotin antibody conjugated to alkaline phosphatase, which will deposit an insoluble indigo color product on the surface of beads bound to the biotinylated target macromolecule. Another way of detecting binding involves looking for fluorescent beads that fluoresce from a cyanine or other dye attached to the target macromolecule.

While current methods of OBOC screening have been successfully used to detect new synthetic compounds or ligands against these target macromolecules, these methods have several shortcomings that limit their effectiveness. For example, the attachment of a label can interfere with binding. The label may sterically block parts of the target macromolecule. Additionally, the process of attaching a label can alter the structure of the target macromolecule and/or result in loss of the target macromolecule due to low yield of label attachment reactions and subsequent purification. There can also be false positive beads resulting from specific binding of the anti-biotin antibody to the compound displayed on the bead, rather than through binding to the bead bound biotinylated target macromolecule (Lam, K. S. et al. (1991) Nature 354:82). False positives also arise from interactions between the label and the dye.

An alternative OBOC screening technique—the Bead Blot—eliminates the use of labels, but it has its own limitations (Lathrop, J. T. et al. (2007) Anal Biochem. 361:65-76). The Bead Blot involves first reacting beads with a target macromolecule, then transferring the target macromolecule to a membrane, and probing the membrane with an antibody or other method. Spots containing the transferred target macromolecule are developed and then matched up with the original gel to determine which beads became bound to the target macromolecule.

In the Bead Blot method, beads are gelled in agarose; this requires the target macromolecule-ligand complexes on each bead to be exposed to warm temperatures that may cause the complex to dissociate. This may be abrogated by screening at warm temperature, but for best results, the screen should be performed at the temperature at which the ligand-target needs to be bound and not at the temperature convenient for the Bead Blot. Agarose gels also pose an issue with handling because they are brittle and easily break. Moreover, the transfer of a target macromolecule to a membrane is carried out via electrophoresis and takes extra time. In particular, the original authors of the Bead Blot recommended using several types of transfer buffers and suggest allowing each type to proceed for a day. As a result, it takes each Bead Blot several days to develop, which significantly hampers the throughput of the screen since the result of a screen will not be known for several days.

Another issue exists with the method for developing the Bead Blot. To develop the blot, the authors of the method introduce antibodies for each target macromolecule they transfer to a membrane, and the antibodies produce chemiluminescent spots on a film that they later align with beads. This method is problematic because some target macromolecules do not have available antibodies necessary to develop in this manner. It is possible to develop using a radiolabelled target macromolecule; however, such an altered target macromolecule may be difficult to produce with some target macromolecules, and synthesizing such a target introduces the shortcomings associated with labels described above. Moreover, autoradiography is not an ideal development technique because, as spots develop, some get so large that they cover other spots of nearby beads, thereby impeding detection efforts.

Accordingly, a significant need exists for improved OBOC assay techniques and related components and kits.

### SUMMARY

In particular, there is a significant need for OBOC assay methods, related compositions for performing the methods, and kits that can achieve high throughput screening without the shortcomings associated with current OBOC techniques. There is a need for screening techniques that allow for efficient and effective detection of compounds such as for example, lead compounds without interference from labels. There is also a need for an OBOC method that is reliable and easy to use. Various aspects disclosed herein fulfill one or more of these needs.

The systems and methods described herein each have several aspects, no single one of which is solely responsible for its desirable attributes. Without limiting the scope of this disclosure as expressed by the claims that follow, the more prominent features will now be discussed briefly. After considering this discussion, and particularly after reading the section entitled “Detailed Description,” one will understand how the sample features described herein provide for improved screening of compound libraries.

Provided herein are methods of detecting a compound that is bound to a target macromolecule, the method comprising, or alternatively consisting essentially of, or yet further consisting of, contacting the compound when attached to a bead, e.g. OCOB (termed a “compound bead” herein) or a plurality of compound beads (e.g., a compound library bound to a plurality of beads, with one compound per bead) with one or more target macromolecules to form a compound-target macromolecule complex or complexes. As known to those of skill in the art, a complex will form between two entities when there is sufficient affinity between the entities. If such an affinity is not present, the target macromolecule will not bind to the compound bead, and unbound target macromolecule and compound beads will remain. However, where all target macromolecules bind to the compound-beads, no unbound target macromolecules or compound beads remain. Thus, in one aspect, the method further comprises the step of separating compound-target macromolecule complex from unbound target macromolecule and/or compound bead to form separated complex.

After or absent the separation of complex from unbound target macromolecule or bead compounds, the complexes of the target macromolecules and compound beads are combined with a separation matrix. Non-limiting examples of a separation matrix are an acrylamide gel, a collagen matrix, and an agarose gel and a non-limiting example of a method of separating the target macromolecules from the separated complex comprises electrophoretic separation.

The method further comprises staining the gel with a stain that is particular for the target macromolecule. Non-limiting examples of staining of the separated complex comprises contacting with a solution elected from the group of silver and a Sybr gold dye. These solutions are commercially available from various vendors.

In another aspect, the step of isolating the separated target macromolecules comprise combining the complexed compound beads with a matrix and subsequently allowing the matrix to form a substantially solid matrix; electrophoretically separating the target macromolecule-compound bead complexes from each other isolating the separated compound beads. Non-limiting examples of appropriate parameters for electrophoreses comprises, or alternatively consists essentially of, or yet further consists of, applying a voltage for about 10 seconds to about 1 hour, and in some such embodiments, it involves applying a voltage for about 15 seconds to about 45 seconds. In some embodiments, a voltage is applied at from about 270V to about 330V, or alternatively from about 300V and at about 8 Amps to 10 Amps and for the times as noted above.

Another aspect of the present disclosure is directed to a method of detecting a compound bound to a target macromolecule. In various embodiments, the method comprises, or alternatively consists essentially of, or yet further consists of: synthesizing a compound library onto beads to form an immobilized compound bead library; introducing a target macromolecule to the immobilized compound library, wherein introducing a target macromolecule is performed under conditions that favor binding of the target macromolecule to one or more of the compounds of the immobilized compound library to produce target macromolecules bound to compound beads if there is binding affinity between one or more of the compounds for the target macromolecule(s); transferring the beads of the complexed, immobilized compound library to an separation matrix such as an acrylamide gel solution; forming a solidified gel comprising the beads of the immobilized compound library set in the acrylamide gel solution; incubating the solidified gel in an electrophoresis buffer; applying a voltage to the solidified gel to perform electrophoresis; and staining the solidified gel to observe one or more positive compound beads. A positive compound bead is a bead of the immobilized library which experienced binding with the target macromolecule and each of the one or more positive compound beads form an observable streak on the gel adjacent to the bead due to the staining of the macromolecule. Because the target macromolecule and not the compound of interest is stained, the integrity and chemical structure of the compound of interest remains intact or unaltered. Non-limiting examples of target macromolecules and/or the compounds of interest include an organic or inorganic small molecule, a peptide, a polypeptide, an antibody, antibody fragment, a nucleic acid (DNA, RNA, synthetic or wild-type) and macromolecules such as a polymer, a glycan, or carbohydrate, fusions of a small molecule or polypeptide with another molecule such as a polymer-polypeptide fusion. For example, in some embodiments, the one or more compounds comprise one or more proteins. In other embodiments, the one or more compounds include one or more nucleic acids. In still other embodiments, the one or more compounds comprise one or more lipids, sugars, or other molecules. For the purpose of this disclosure, the compounds within a compound bead library may be identical or different. The target macromolecule can be isolated or a component of another entity, e.g., a protein marker present on the surface of a cell, e.g. an eukaryotic or prokaryotic cell such as a cancer cell or an a prokaryotic cell such as E coli.

In at least some embodiments of the method, introducing one or more target macromolecules to the immobilized compound library comprises, or alternatively consists essentially of, or yet further consists of, incubating the compound library in a solution containing the one or more target macromolecules.

In various embodiments, the method also further comprises, or alternatively consists essentially of, or yet further consists of, removing the unbound target macromolecules, for example, by washing the compound beads to remove unbound target macromolecules prior to transferring the compound beads to the gel solution. A non-limiting example of forming a solidified gel comprises, or alternatively consists essentially of, or yet further consists of: transferring by e.g., pipetting the acrylamide gel solution with the beads of the immobilized compound library onto a solid surface such as a gel plate, and allowing the the gel solution to set or form a substantially solidified gel. In various embodiments, incubating the solidified gel in a suitable solution, e.g., an electrophoresis buffer, causes the gel to equilibrate and causes the target macromolecule to denature and develop a negative charge.

In some embodiments of the method, an electronic voltage is applied to the solidified gel to separate the beads to perform electrophoresis. Non-limiting examples of appropriate parameters for electrophoreses comprises, or alternatively consists essentially of, or yet further consists of, applying a voltage for about 10 seconds to about 1 hour, and in some such embodiments, it involves applying a voltage for about 15 seconds to about 45 seconds. In some embodiments, a voltage is applied at from about 270V to about 330V, or alternatively from about 300V and at about 8 Amps to 10 Amps and for the times as noted above.

The method of some embodiments, further comprises, or alternatively consists essentially of, or yet further consists of, placing the solidified gel in a solution to fix the target macromolecule and remove electrolytes prior to staining. Any suitable stain may be used to directly or indirectly stain the gel. For example, in some embodiments, staining the solidified gel comprises incubating the solidified gel in a silver nitrate solution. In some other embodiments, staining the solidified gel comprises incubating the solidified gel in a Sybr gold dye.

The method may further comprise, or alternatively consist essentially of, or yet further consist of, removing the one or more compound beads identified as “positive” (by noting the positive stain or comet in the gel), from the gel and identifying and in some appropriate aspects the isolated compound. For example, one can sequence the compounds after isolation of the compounds from the gel. In some such embodiments, removing the one or more positive compound beads from the gel comprises, or alternatively consists essentially of, or yet further consists of separating by cutting or incising the one or more positive compound using any appropriate method or tool. A non-limiting example comprises the use of a gauge needle, and extracting the compound-bead with a pipette. In some embodiments, sequencing is in the isolated compound bead performed by an automated Edman-degradation microsequencer.

Another aspect of the present disclosure is directed to a kit for detecting a compound that binds a target macromolecule. The kit of various embodiments includes: one or more components needed to form an acrylamide gel; one or more components needed to form an electrophoresis buffer; and one or more components needed to directly stain the acrylamide gel and instructions for use. In addition, an immobilized compound library or one or more components needed to form an immobilized compound library is provided in the kit.

In some embodiments, the one or more components needed to form an immobilized compound library comprise a plurality of beads and/or a solution for containing one or more compound bead libraries and optionally target macromolecules. In some embodiments, the one or more components needed to form an acrylamide gel comprise one or more of: L-Serine, L-Glycine, L-Asparagine, and acrylamide. In some embodiments, the one or more components needed to form an electrophoresis buffer comprise one or more of: a Laemmli buffer, a TBE running buffer, urea, and SDS. In some embodiments, the one or more components needed to directly stain the acrylamide gel comprise components of a proteosilver kit or components of a Sybr Gold dye kit. Moreover, the kit of some embodiments further includes one or more gel plates and/or one or more filter paper wicks.

Those skilled in the art will appreciate that the foregoing is a summary and thus, contains by necessity, simplifications and omissions of detail. Any particular device or method may have some of or all these features or additional or alternative features. Other aspects, features, and advantages will become apparent in the teachings that follow.

### BRIEF DESCRIPTION OF THE DRAWINGS

The above-mentioned aspects, as well as other features, aspects, and advantages of the present technology will now be described in connection with various embodiments, with reference to the accompanying drawings.

FIG. 1 is a photograph of a top perspective view of one embodiment of a gel prior to electrophoresis; the gel is set up in accordance with the description provided herein.

FIGS. 2A-2B are photographs of gels following electrophoresis performed in accordance with the principles of the present technology. Specifically, FIGS. 2A-2B depict experimental results of a control experiment. The gel of FIG. 2A includes E. coli-binding beads with a peptide of the amino acid sequence “klavr” incubated with E. coli in 1×PBS. The gel of FIG. 2B includes just PBS with no E. coli.

FIGS. 3A-3D are photographs of gels following electrophoresis performed in accordance with the principles of the present technology. Specifically, FIGS. 3A-3D depict results of an experiment designed to compare the effects of varying gel incubation times in an electrophoresis buffer.

FIGS. 4A-4D are photographs of gels following electrophoresis performed in accordance with the principles of the present technology. Specifically, FIGS. 4A-4D depict results of an experiment designed to compare the effects of varying electrophoresis running time.

FIGS. 5A-5B are photographs of a gel following electrophoresis performed in accordance with the principles of the present technology. Specifically, FIG. 5A depicts the results of assaying a random peptide library binding Human Serum Albumin. FIG. 5B provides a zoomed in image of a positive bead found in FIG. 5A.

FIGS. 6A-6B are photographs of a gel following electrophoresis performed in accordance with the principles of the present technology. Specifically, FIG. 6A depicts the results of a zinc finger-like peptide library binding to Molt4 cytoplasmic proteins. FIG. 6B provides a zoomed in image of a positive bead found in the top right of FIG. 6A.

FIGS. 7A-7B are photographs of a gel following electrophoresis performed in accordance with the principles of the present technology. Specifically, FIG. 7A depicts the results of a zinc finger-like peptide library binding to plasma proteins. FIG. 7B provides a zoomed in image of a positive bead found in the top center of FIG. 7A.

FIGS. 8A-8B are photographs of gels following electrophoresis performed in accordance with the principles of the present technology. Specifically, FIG. 8A depicts the results of assaying a small molecule Actinomycin D-based library with tRHA/miRNA-34a chimera, and FIG. 8B depicts the results of an 8-amino acid linear library screened with salmon sperm DNA.

FIG. 9 is a photograph of a gel following electrophoresis performed in accordance with the principles of the present technology. The photograph was taken with an Olympus fluorescent microscope with a movable stage using a filter assigned for cy3. Tentagel resin auto-fluoresces at this wavelength, and hence, beads are also visible. The photograph depicts the results of a random 12 amino acid linear library screened against salmon sperm DNA fragments stained with SYBR gold dye.

FIG. 10 is a photograph of a gel following electrophoresis performed in accordance with the principles of the present technology. Specifically, FIG. 10 depicts the results of assaying a peptide sequence bound to molt4 cytoplasmic proteins.

### DETAILED DESCRIPTION

In the following detailed description, reference is made to the accompanying drawings, which form part of the present disclosure. The embodiments described in the drawings and description are intended to be exemplary and not limiting.

### Definitions

As used herein, the term “exemplary” means “serving as an example or illustration” and should not necessarily be construed as preferred or advantageous over other embodiments. Other embodiments may be utilized and modifications may be made without departing from the spirit or the scope of the subject matter presented herein. Aspects of the disclosure, as described and illustrated herein, can be arranged, combined, and designed in a variety of different configurations, all of which are explicitly contemplated and form part of this disclosure.

Unless otherwise defined, each technical or scientific term used herein has the same meaning as commonly understood by one of ordinary skill in the art to which this disclosure belongs. In accordance with the claims that follow and the disclosure provided herein, the following terms are defined with the following meanings, unless explicitly stated otherwise.

The term “about,” “approximately,” or “substantially” when used before a numerical designation or range (e.g., pressure or dimensions), indicates approximations which may vary by (+) or (−) 5%, 1% or 0.1%.

As used in the specification and claims, the singular form “a”, “an” and “the” include both singular and plural references unless the context clearly dictates otherwise. For example, the term “a macromolecule” may include, and is contemplated to include, a plurality of macromolecules. At times, the claims and disclosure may include terms such as “a plurality,” “one or more,” or “at least one;” however, the absence of such terms is not intended to mean, and should not be interpreted to mean, that a plurality is not conceived.

A “plurality,” as used herein, shall refer to two, three, four, five, or more.

As used herein, the term “comprising” or “comprises” is intended to mean that the kits, systems, and methods include the recited elements, and may additionally include any other elements. “Consisting essentially of” shall mean that the kits, systems, and methods include the recited elements and exclude other elements of essential significance to the combination for the stated purpose. Thus, a kit or method consisting essentially of the elements as defined herein would not exclude other materials or steps that do not materially affect the basic and novel characteristic(s) of the claimed invention. “Consisting of” shall mean that the kits, systems, and methods include the recited elements and exclude anything more than a trivial or inconsequential element or step. Embodiments defined by each of these transitional terms are within the scope of this disclosure.

“Screening” methods or techniques, as used herein, shall refer to any approach for determining which, if any, compounds within a combinatorial library bind to a target macromolecule.

“One-Bead-One-Compound” methods or techniques, also referred to “OBOC” methods or techniques, shall refer to any screening method or technique in which the library is formed of a plurality of compounds, each compound bound to a separate bead.

“Lead compounds,” as used herein, shall mean pharmacologically or other chemically promising compounds.

As used herein, the term “compound” and/or “compound of interest” intends include for example, any molecule or entity that has the ability to form a complex with another entity. Non-limiting examples of compounds include a small organic or inorganic molecule, a peptide, a polypeptide, an antibody, antibody fragment, a nucleic acid (DNA, RNA, synthetic or wild-type) and macromolecules such as a polymer, a glycan, or carbohydrate, fusions of a small molecule or polypeptide with another molecule such as a polymer-polypeptide fusion. For example, in some embodiments, the one or more compounds comprise one or more proteins. In other embodiments, the one or more compounds include one or more nucleic acids. In still other embodiments, the one or more compounds comprise one or more lipids, sugars, or other molecules. For the purpose of this disclosure, the compounds in the method may be identical or different entities.

A “compound bead,” as used herein, shall refer to a bead bound to a compound.

A “positive bead,” as used herein, shall refer to a compound bead bound to a target macromolecule.

“Bead-comet assay” shall refer to various embodiments of the screening techniques and technologies introduced herein, the details of which are provided below.

As used herein, the terms compound includes for example, any molecule or entity that has the ability to form a complex with another entity. Non-limiting examples of compound and/or compound of interest include an organic or inorganic small molecule, a peptide, a polypeptide, an antibody, antibody fragment, a nucleic acid (DNA, RNA, synthetic or wild-type) and macromolecules such as a polymer, a glycan, or carbohydrate, fusions of a small molecule or polypeptide with another molecule such as a polymer-polypeptide fusion. For example, in some embodiments, the one or more compounds comprise one or more proteins. In other embodiments, the one or more compounds include one or more nucleic acids. In still other embodiments, the one or more compounds comprise one or more lipids, sugars, or other molecules. For the purpose of this disclosure, the compounds within a compound bead library may be identical or different.

As used herein, the term “introducing” intends placing two or more entities in close proximity with each other, e.g., contacting. In some aspect, the entities are introduced so that a covalent or other strong chemical or electrostatic bond can form between the two or more entities, e.g., the target macromolecule and the compound.

To “separate” intends removing from close proximity two or more entities that have not formed a covalent or other strong chemical or electrostatic bound with each other.

### Modes for Carrying Out the Disclosure

While there are several methods available to screen compounds, they all have significant limitations, as mentioned above. The label-based methods are not as effective as desired. For example, a label on a target molecule can interfere with the binding of a target macromolecule to that target. Additionally, the label can cause spurious binding that does not involve, or partially involves, the target. The label may require attachment by a process that alters the target, and/or require a difficult process to attach. The known label-free OBOC screening method, the Bead Blot, has its own issues. For example, it is difficult to perform the method successfully. With the method, there is a need to accurately align the membrane to identify positive beads, and it is difficult to develop the film to show the macromolecules transferred to the membrane. Moreover, the method requires the use of agarose gels, which are brittle and fragile, and requires significant time (e.g., several days) to transfer proteins or other macromolecules to the membrane via capillary action.

Knowing the limitations of existing methods, the authors of the present disclosure developed a new label-free method for OBOC screening, described herein. The method, termed bead-comet assay, is capable of staining proteins and/or other molecules coming off the beads directly in the gel, using polyacrylamide gels instead of agarose. The bead-comet assay described herein is substantially different than the conventional Comet Assay, which is a label-free technique that involves staining the contents of cells to detect DNA damage in single cells. In the conventional Comet Assay, DNA damage is detected by viewing a fluorescent comet (Singh, N. P. et al. (1988) Exp. Cell Res. 175:184-191) originating from those cells in an agarose gel; the comet has a head of intact DNA and the tail is composed of smaller damaged DNA. Notably, the existing assay is only used to detect DNA in cells, has no known application in combinatorial chemistry, and uses agarose gels.

Provided herein are methods of detecting a compound that was bound to a target macromolecule. The method requires contacting the compound that is attached to a bead or a plurality of compounds attached to a plurality of beads, e.g., OCOB, with one or more target macromolecules to form a complex if there is affinity for the compound and the target macromolecule. The complex is mixed with a separation matrix and separating the complexes from the target macromolecules. This can be accomplished by applying a current to separate the target macromolecule from the compound. The target macromolecule is stained and the compound adjacent to the stain can be isolated by removal from the gel. As known to those of skill in the art, a complex will form between two entities when there is sufficient affinity between the entities, non-limiting examples of such include a covalent bond or a strong electrochemical bound (e.g., a streptavidin-biotin bond). If such an affinity is not present, the target macromolecule will not bind to the compound, and unbound target macromolecule will remain. However, where all target macromolecules bind to the compound, no unbound target macromolecule remains. Thus, in one aspect, the method further comprises the step of separating compound-target macromolecule complexes from unbound target macromolecule to form separated complexes prior to combining separated complex with the separation matrix. In another aspect, the method further comprises synthesizing the compound bead library by contacting a plurality of compounds with a plurality of beads under conditions to bind the plurality of compounds to the beads. Methods to bind compounds to beads are known in the art.

Non-limiting examples of a separation matrix are an acrylamide gel, a collagen matrix, and an agarose gel and a non-limiting example of a method of separating the target macromolecules from the separated complex comprises electrophoretic separation.

Non-limiting examples of staining of the separated complex comprises contacting with a solution elected from the group of silver or a Sybr gold dye. These solutions are commercially available from various vendors.

In another aspect, the step of isolating the separated target macromolecules comprise combining with a matrix and subsequently allowing the matrix to form a substantially solid matrix; electrophoretically separating the target macromolecules in the matrix; and isolating the separated target macromolecules.

In some embodiments of the method, an electronic voltage is applied to the solidified gel to separate the beads to perform electrophoresis. Non-limiting examples of appropriate parameters for electrophoreses comprises, or alternatively consists essentially of, or yet further consists of, applying a voltage for about 10 seconds to about 1 hour, and in some such embodiments, it involves applying a voltage for about 15 seconds to about 45 seconds. In some embodiments, a voltage is applied at from about 270V to about 330V, or alternatively from about 300V and at about 8 Amps to 10 Amps and for the times as noted above.

Using the present bead-comet assay described herein, target macromolecules that are bound to compound beads can be directly detected within a gel without the use of labels. Specifically, target macromolecules are stained directly to enable detection. In various embodiments, after binding to the target macromolecule, beads are gelled within a polyacrylamide gel, and electrophoresis is applied to pull the target off the beads after denaturation. Target macromolecules migrate just a short distance from the respective bead to which each was bound, and said molecules are visible adjacent to said beads as streaks of staining within the gel.

Advantageously, the presently described assay is simple to perform, can be done in one day, and has been demonstrated for a broad variety of target macromolecules such as protein based target macromolecules and nucleic acid based target macromolecules, and with the potential for screening lipid and sugar based target macromolecules too. Additionally, being label-less has several advantages, including, for example, the following:

1) The potential of false positive interactions between a test ligand (e.g., a small molecule compound) and a label present on a target macromolecule is eliminated. This assay gives accurate binding results.

2) The labelling process is eliminated; the labelling process can involve chemical modifications that may deform the target macromolecule.

3) It would spare precious samples from having to be labelled, preserving them for future studies.

4) Time and effort devoted to labelling a target macromolecule is eliminated.

In some embodiments, the assay is used to identify promising proteins. In one embodiment of the method, compound beads—some of which are bound to selected target proteins—are put into a polyacrylamide gel, and the bound proteins are pulled off the beads using PAGE gel electrophoresis (Laemmli, U. K. (1970) Nature 227(5259):680-685). In various embodiments, polyacrylamide gels are used rather than agarose, because polyacrylamide gels are not brittle or fragile and they are easy to use. The polyacrylamide gels overcome the issues associated with agarose gels described above, and the properties of polyacrylamide gels (such as pH and pore size) can be well controlled. In various embodiments, the pH of the polyacrylamide gels is kept at the same pH as the binding condition, e.g., pH 7.5, to prevent early dissociation of target molecules from beads into the gel. Gels of various embodiments are incubated in a denaturing running buffer that denatures the ligand-target macromolecule complexes formed, and in the case of proteins, provides negative charges for electrophoresis.

In one embodiment, silver staining is used to detect the macromolecules coming from the beads. Silver staining is preferred in some embodiments due to its superior sensitivity and ability to stain a variety of different molecules (Chevallet, M. et al. (2006) Nature Protocols 1:1852-1858). In other embodiments, any other suitable stain may be used. For example, in some embodiments, a fluorescent stain is used. Such a stain may include, but is not limited to, Sybr Gold.

In various embodiments of the method, macromolecules pulled off the beads appear as one or more band(s) or a smear (i.e., comet) of staining adjacent to a positive bead. The positive beads are thus, detectable. In some embodiments, the positive beads are detected, taken, and/or successfully identified with a computerized sequencer or analyzer, such as, for example, an Edman degradation micro-sequencer to determine their sequence.

In another embodiment, the assay is used to identify promising nucleic acid target macromolecules. In still other embodiments, the assay described above is used to identify other promising macromolecule targets suitable for use as a pharmaceutical, a biopharmaceutical, or other chemical applications.

Also provided is a kit for detecting a compound, the kit comprising, or alternatively consisting essentially of, or yet further consisting of, one or more components to form an substantially solid matrix; and one or more components to stain the matrix. In one aspect, the kit further comprises, or alternatively consisting essentially of, or yet further consisting of, a immobilized compound library comprising a plurality of beads and/or a plurality of beads to prepare a compound bound to a bead. In one aspect, the one or more components needed to form a substantially solid matrix comprise an acrylamide gel. In another aspect, the kit further comprises or alternatively consists essentially of, or yet further consists of, one or more components to form an electrophoresis buffer, optionally comprising one or more of: a Laemmli buffer, a TBE running buffer, urea, and SDS. Non-limiting examples of the one or more components to stain the matrix comprises components of a proteosilver kit or components of a Sybr Gold dye kit. In a further aspect, the kit further comprises, or alternatively consists essentially of, or yet further consists of, one or more gel plates and one or more filter paper wicks. In another aspect, the kit further comprises, or alternatively consists essentially of, or yet further consists of instructions to perform a methods as disclosed herein.

The following examples are provided to exemplify and not limit the invention.

### EXAMPLES

### Experimental Setup

For the various experiments performed, random amino acid OBOC libraries were synthesized on tentagel using known procedures.

In a first set of experiments, protein incubation involved transferring 50 μL of beads into a 2 mL column. The bead-transfer solution was drained via a vacuum and replaced with protein containing solution usually in PBSTN (PBS with between 0.1% and 0.01% sodium azide as a preservative). The beads were then rotated in the protein containing solution for 1 hour. After 1 hour, the protein solution was drained and the beads were washed 3 times with PBSTN and then beads were rotated in PBSTN for 1 hour to remove loosely bound proteins from the beads. Beads were then washed 3 times with water to remove PBSTN.

A gelling step was also performed. Beads were transferred to acrylamide gel solution formulated based on the teachings of Ahn, T. et al. (Ahn, T. et al. (2001) Anal Biochem. 291:300-303), containing 0.1 M L-Serine (Irvine Scientific 96813), 0.1 M L-Glycine (Sigma G8898), 0.1 M L-Asparagine (Sigma A4159), 0.025 M Tris (Sigma T1503), and 12% Acrylamide (BioRad 161-0107). Tris was added to the gel solution from a 20× stock solution prepared at pH 7.4 to prevent proteins from dissociating from beads before the gel solidified. After beads were transferred to the gel solution, 10% Ammonium Persulfate (Sigma A3678) in milliQ water was added as well as TEMED (Sigma T8133), then the gel solution with the beads was pipeted into the gel plates to create the gel. Because the beads fall to the bottom of the gel if the gel plates are kept upright, the plates needed to be turned horizontally to keep the beads properly distributed. The gel with beads was allowed to set for 1 hour.

The gel with the beads was removed from the gel plates and incubated in the electrophoresis buffer for 30 minutes to allow the gel to equilibrate, the proteins to denature, and to give the proteins negative charge with SDS. The electrophoresis buffer was the standard laemli buffer (Laemmli, U. K. (1970) Nature 227(5259):680-685) with 1% SDS (Sigma L3771). Two filter paper wicks are incubated in electrophoresis buffer beforehand as well. After incubating the gel in electrophoresis buffer, the gel was placed on a glass gel plate, then the glass plate was placed in the center of a horizontal electrophoresis machine. Filter paper wicks were placed on the edge of the gel for each electrode solution. The wicks were placed as close to the edge as possible to enable part of the gel to be cut off afterwards; without such cutting, it may stain very dark. A photograph showing a top perspective view of an exemplary gel setup with wicks before electrophoresis is provided in FIG. 1.

Once set up, the electrophoresis was carried out for 30 seconds at 300V and around 8 to 10 Amps. The time required to pull protein off the beads may vary based on the protein of interest, but this condition worked well for the protein types tested.

After electrophoresis, the gel was placed in a fixing solution to fix the proteins in place and remove electrolytes. Fixation is done in 10% acetic acid (Sigma Aldrich A9967) and 50% ethanol (Sigma Aldrich 459844) in water for two changes either 30 min each, or in some embodiments, it was done overnight. After fixing, for convenience, a proteosilver kit (Sigma Aldrich product number Protsill) was used and the kit instructions were followed with one important difference—during the silver nitrate addition step, instead of allowing the gel to incubate for 10 minutes, the gel was allowed to incubate for 1 hour. Alternatively, in other embodiments and experimental setups, the protocol in (Protocol for Silver Staining of Gels Optimized for Mass Spectrometry and Protein Identification. Retrieved from: alphalyse.com/fileadmin/Alphalyse/PDF/Silver_staining_protocol.pdf) was used. The gel was incubated in the silver nitrate solution for 1 hour to allow more time for a latent image to form before the gel was developed. The beads in the gel, which stain on their own without proteins due to the peptides on their surfaces, have darker staining when the silver nitrate step is prolonged. Moreover, prolonging the silver nitrate step yields better staining of the proteins coming off the beads.

In a second set of experiments, all steps of the assay process were the same as described above for the first set of experiments, with the following exceptions: polyacrylamide gels were 6% acrylamide and composed of 1× TBE buffer, pH 7.5; prior to electrophoresis, gels were incubated with 1× TBE running buffer containing 6M urea and 1% SDS to denature nucleic acids; and after electrophoresis, staining was achieved using Silver Stain following the protocol found in Bassam et al. (Lathrop, J. T. et al. (2007) Anal Biochem. 361:65-76), or using Sybr Gold dye following instructions from Invitrogen.

In both the first and second set of experiments, the beads were cut out of the gel using a 20 gauge needle then sucked up using a pipette. Beads were washed 3× with water and then treated with 100% TFA for 1 hour. TFA beads were transferred onto filter paper for sequencing. Beads were sequenced using an automated edman-degradation microsequencer.

The following examples are provided to illustrate, and not limit the invention of the claims.

### Example 1

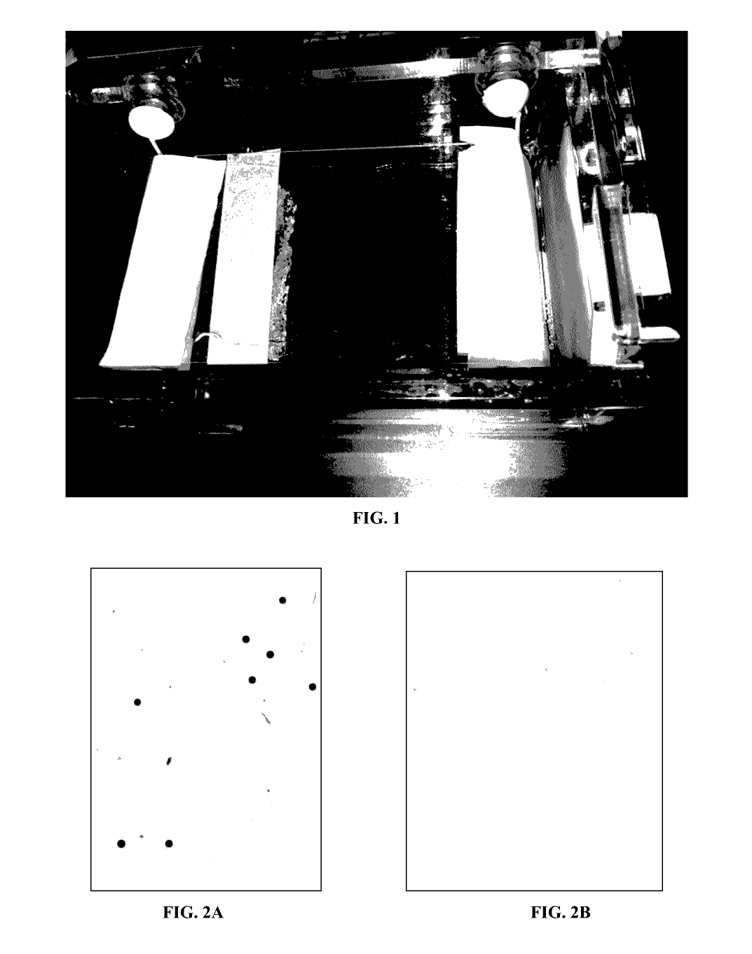

In a first example, a known sequence was tested against Escherichia Coli to see if the improved OBOC screening methods described herein were feasible against bacterial cells. As shown in FIGS. 2A-2B, all the beads had a streak of staining coming off them that suggested binding when the beads were incubated with E. coli, but none of the beads had streaks without E. coli.

### Example 2

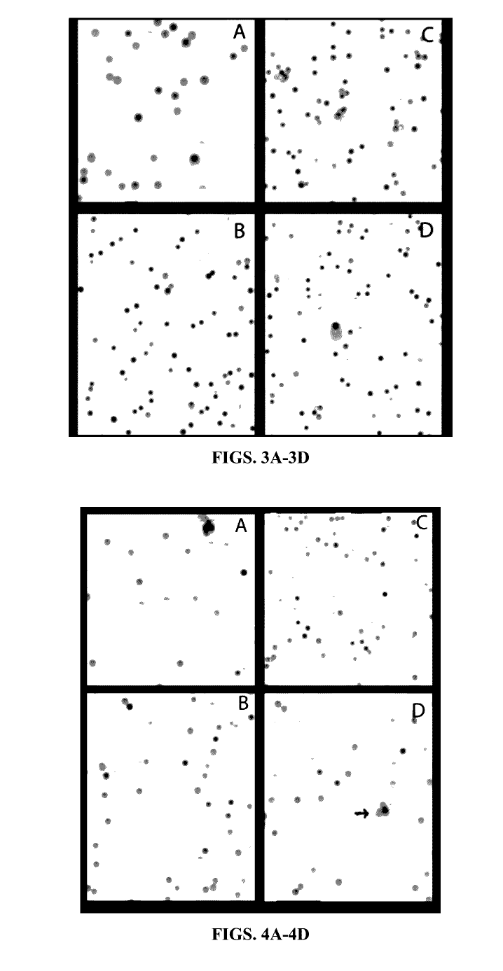

After finding streaks coming off E. coli binding beads, a second set of examples was performed using protein mixtures and individual proteins. As shown in FIGS. 3A-3D and 4A-4D, care was taken to find the amount of time required to denature serum proteins bound to beads (FIGS. 3A-3D) and the amount of time required to pull those serum proteins off the bead to produce visible staining (FIGS. 4A-4D). Beads were incubated with serum proteins and gelled, as described above. The gel was cut into four sections and electrophoresis was performed after incubating the gel. In the experiments of FIGS. 4A-4D, electrophoresis was kept at 300V to move the proteins off the beads in a short amount of time, and the time needed to pull bound serum proteins off the beads at that voltage was tested. Electrophoresis was performed for 10 seconds in FIG. 4A, 30 seconds in FIG. 4B, 1 minute in FIG. 4C, and 5 minutes in FIG. 4D. It was found that 30 seconds was the best condition for serum proteins because shorter time gave shorter comets and longer time gave longer ones. The comets got even longer with longer time in our test rather than just separating from the beads.

When we tested the same serum proteins varying the amount of time sitting in SDS running buffer, we found that by 10 minutes, serum proteins were able to be pulled off the beads and longer times may not be necessary. There was also no impact on the shape of the comets when time in SDS running buffer was increased to 2 hours. As shown in the experiments of FIGS. 3A-3D, the gels were incubated in SDS running buffer for 10 minutes in FIG. 3A, 30 minutes in FIG. 3B, 60 minutes in FIG. 3C, and 120 minutes in FIG. 3D.

### Example 3

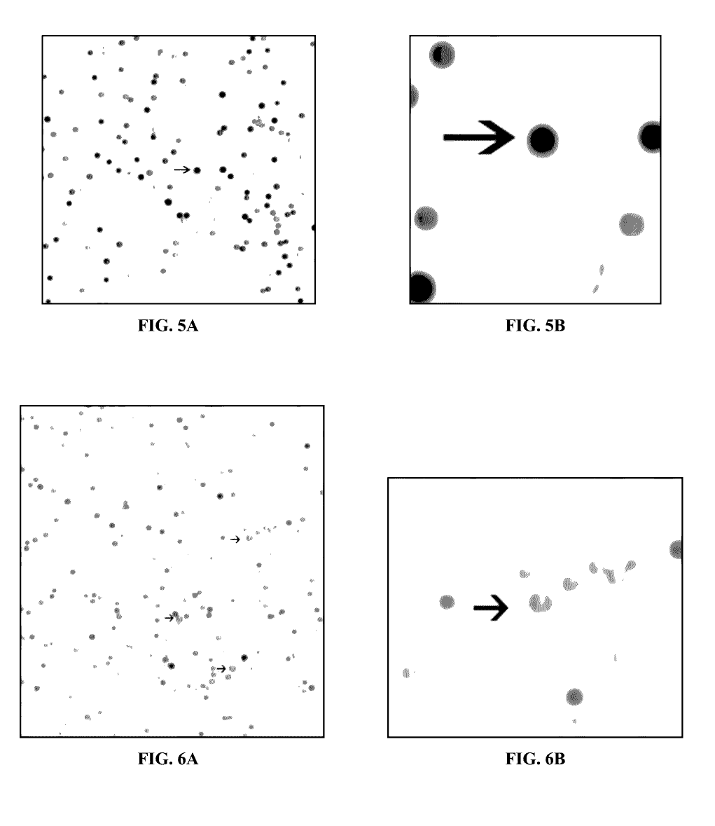

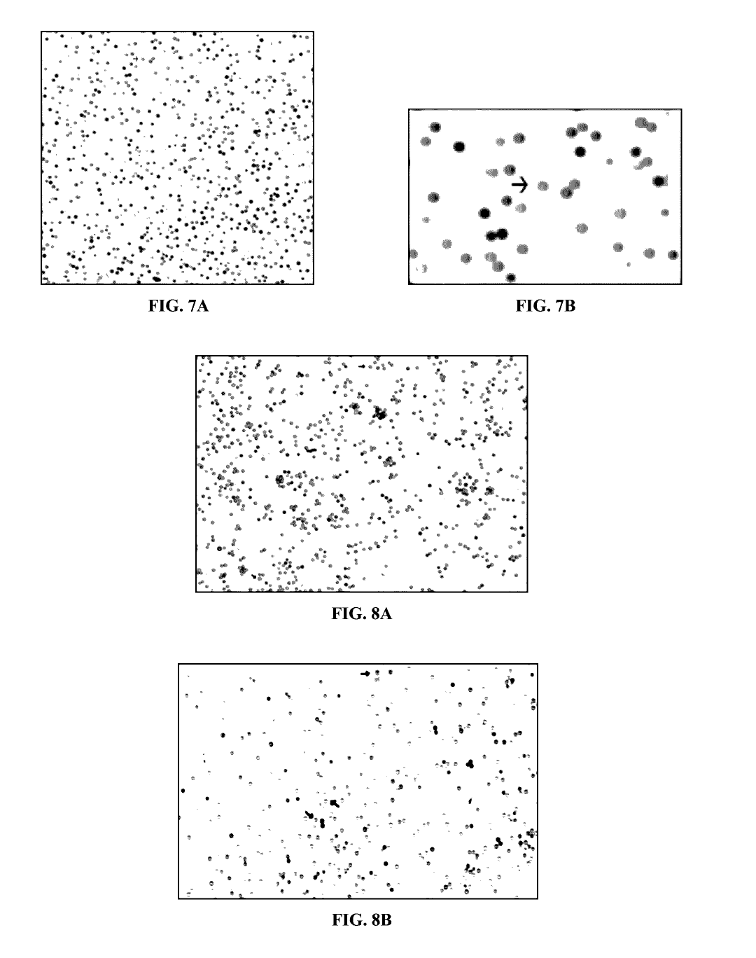

Different protein types were also tested, using 30 minutes of incubation in SDS running buffer and 30 to 45 seconds running time of electrophoresis at 300V as a benchmark. In particular, proteins tested include: Human Serum Albumin (HSA) (see FIGS. 5A-5B), proteins extracted from Molt4 cytoplasm (see FIGS. 6A-6B), and plasma proteins (see FIGS. 7A-7B). In FIGS. 5A-5B, 6A-6B, 7A-7B, red arrows highlight positive beads. This technique worked to find beads with streaks or “comets” for each type of protein. Of interest is that HSA also showed a comet appearance instead of a single band, even though it is not a mixture of proteins like the others.

### Example 4

This method has also proven useful for nucleic acids. In another example, as shown in

FIGS. 8A-8B, DNA and RNA molecules were screened and stained using TBE based polyacrylamide gels instead of Tris based. When tested, DNA and RNA based target macromolecules also showed comets.

### Example 5

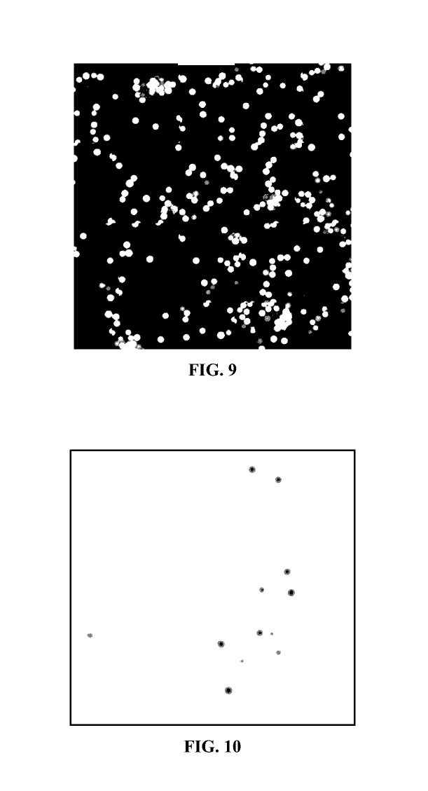

In addition to silver staining, fluorescent dyes were tested and found to work well. FIG. 9 shows salmon sperm DNA comets stained with SYBR Gold dye.

### Example 6

Several peptide beads that were found to bind to Molt4 proteins were remade, and when retested, showed comets again (see FIG. 10). Some of the beads broke when synthesizing this peptide, and under the microscope, it can be seen that the bead fragments had comets that were proportionally smaller than intact beads. Three broken beads are highlighted by red arrows.

### Discussion

Using the improved OBOC screening method disclosed herein, it is possible to view target macromolecules coming off of an OBOC bead, as evidenced by the comets visible in the gels adjacent to the beads. These comets were shown to be the target macromolecule of interest given that comets were not observed in gels that did not include the target macromolecule. The comets were repeatable; when positive beads were resynthesized and retested, the resynthesized beads also showed comets for all beads with the same sequence. The appearance of comets instead of bands for even individual proteins is interesting and suggests that the conditions are not ideal to generate clear cut bands. With different conditions it may be possible to generate individual bands for each protein coming off a positive bead. This method is versatile and can detect target macromolecule such as those found in plasma, bacteria, and to viral particles. Detection of proteins, DNA, and RNA has been shown in the exemplified embodiments. In other embodiments, detecting binding of lipids, sugars and various other molecules may be performed using this method. For best results, care may need to be exercised to limit the presence of contaminants in a OBOC library. Small hairs and pieces of dust, if introduced into the gels, may be stained and show comets. However, there are false positives to any method and true comets that are strong should be able to be distinguished. Accordingly, the disclosed assay accomplishes the goal of creating a high throughput label free assay for detection of new OBOC ligands, which is useful against a wide range of target macromolecules.

### Equivalents

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

What is claimed is:

1. A method of detecting a compound comprising:
contacting a compound bead library with one or more target macromolecules to form a compound-target macromolecule complex;
combining the compound-target macromolecule complex with a separation matrix;
separating the target macromolecules from the compound-target macromolecule complexes;
staining separated target macromolecules; and
isolating the compound.

2. The method of claim 1, further comprising separating compound-target macromolecule complex from unbound target macromolecule to form separated complex prior to combining the complex with the separation matrix.

3. The method of claim 1, further comprising synthesizing the compound bead library by contacting a plurality of compounds with a plurality of beads under conditions to create the complex.

4. The method of claim 1, wherein the separation matrix is a matrix from the group of an acrylamide gel, a collagen matrix, and an agarose gel.

5. The method of claim 1, wherein the separating the target macromolecules from the separated complex comprises electrophoretic separation.

6. The method of claim 1, wherein the staining of the separated complex comprises contacting with a solution silver or a Sybr gold dye.

7. The method of claim 1, wherein the compound or target macromolecule is one or more of a lipid, a sugar, an organic or inorganic small molecule, a polypeptide, a protein, a polypeptide, a polynucleotide, an antibody, an antibody fragment or a combination thereof.

8. The method of claim 1, wherein the step of electrophoretically separating the target macromolecules from the compounds comprises applying a voltage to the matrix for a time from about 10 seconds to about hour.

9. The method of claim 8, wherein the step of electrophoretically separating the target macromolecules from the beads comprises applying a voltage to the matrix for a time from about 15 to about 45 seconds.

10. The method of claim 8, wherein applying the voltage comprises applying 300V at about 8 to 10 Amps.

11. The method of claim 1, further comprising placing the matrix in a fixing solution to fix the target macromolecules to remove electrolytes prior to staining.

12. The method of claim 1, wherein the method is conducted as a high-throughput screening method.

13. The method of claim 1, further comprising identifying the sequence or structure of the compound.

14. The method of claim 12, wherein the compound comprises a polynucleotide and the step of identification comprises polynucleotide sequencing technology.

15. A kit for detecting a compound, comprising:
one or more components to form an substantially solid matrix; and
one or more components to stain the matrix.

16. The kit of claim 15, wherein the kit further comprises a immobilized compound library comprising a plurality of beads and/or a plurality of beads to prepare a compound bound to a bead.

17. The kit of claim 15, wherein the one or more components needed to form a substantially solid matrix comprise an acrylamide gel.

18. The kit of claim 15, further comprising one or more components to form an electrophoresis buffer, optionally comprising one or more of: a Laemmli buffer, a TBE running buffer, urea, and SDS.

19. The kit of claim 15, wherein the one or more components to stain the matrix comprises components of a proteosilver kit or components of a Sybr Gold dye kit.

20. The kit of claim 15, further comprising one or more gel plates and one or more filter paper wicks.
